(19) United States
(12) Patent Application Publication
(10) Pub. No.: US 2026/0355013 A1
(43) Pub. Date: Nov. 12, 2026

(54) AN APPARATUS FOR INSERTING CONTACT LENSES

(71) Applicant: Niall Austin, Lurgan (IE)

(72) Inventor: Niall Austin, Lurgan (IE)

(21) Appl. No.: 19/139,185

(22) PCT Filed: Dec. 14, 2023
(86) PCT No.: PCT/EP2023/085915

§ 371 Date: Jun. 13, 2025

(30) Foreign Application Priority Data
Dec. 14, 2022 (GB) 2218847.8
Aug. 16, 2023 (GB) 2312515.6

(51) Int. Cl. A61F 9/00

(52) U.S. Cl. CPC A61F 9/0061

(57) ABSTRACT

This invention relates to an apparatus (1, 31) for inserting contact lenses comprising a body (3) configured to be releasably mounted on a user's finger, the body having a resiliently deformable arm (9) extending outwardly from the distal end of the body, and the arm having a hand (11) at its outermost end. The hand (11) has an inner surface (13) that is concave-shaped and a drainage through-hole (15) extending through the hand. In use, a user picks a contact lens up direct from its package on the hand of the apparatus. The contact lens will be held there in position on the hand of the apparatus. The user then places the contact lens against their eye and under a small applied force, the contact lens will be released onto the eyeball. This is a simple and effective way to place contact lenses correctly.

### TECHNICAL FIELD

This invention relates to an apparatus for inserting contact lenses.

### BACKGROUND ART

Contact lenses are commonly placed onto the surface of the eye from the tip of a user's finger. However, there are numerous problems with this technique. First of all, this technique requires contact between the user's fingertip and the contact lens before the contact lens is inserted into the eye. Indeed often, the contact lens is placed in the palm of the user's hand before being placed onto the fingertip prior to insertion of the lens. As a consequence, there is a strong possibility over time that the user will develop an eye infection. Another problem with this technique is that it can be difficult, particularly for those inexperienced users of contact lenses or those with unsteady hands, to correctly place the lens in the eye. A further problem experienced by many users of contact lenses is that a large number, in some cases up to 30% of the lenses, are lost by being dropped prior to insertion. It is not uncommon for the lens to fall off the tip of the users finger.

Various devices have been devised to assist in the placement of contact lenses. However, to date, none of the proposed solutions have been deemed entirely satisfactory. Often, these devices still require the contact lens to be placed in the user's hand before it is “picked up” by the placement device for placement onto the wearer's eye. This has the same hygiene drawbacks as placing the contact lens on the eye from the tip of one's finger. In addition, some of the known offerings are relatively complex to manufacture and/or unwieldy to use.

U.S. Pat. No. 3,132,887 in the name of Martinez discloses an applicator for contact lenses that is relatively compact. US Patent No. U.S. Pat. No. 4,200,320 in the name of Durham discloses a contact lens applicator with an alternative construction.

It is an object of the present invention to provide an apparatus for inserting contact lenses that overcomes at least some of the problems with the known offerings. It is a further object of the present invention to provide an apparatus for inserting contact lenses that is simple to use, inexpensive to manufacture, and that offers a useful choice to the consumer.

### SUMMARY OF INVENTION

According to the invention there is provided an apparatus for inserting contact lenses comprising a body configured to be releasably mounted on a user's finger, the body having a resiliently deformable arm extending outwardly from the distal end of the body, the arm having a hand at its outermost end, the hand having an inner surface that is concave shaped and a drainage through-hole extending through the hand.

By having such an apparatus, the user will be able to place the lens on their eyeball with ease. The user will be able to scoop the lens out of its packaging with the apparatus and will not have to make contact with the lens with their fingertip. Therefore, the likelihood of getting an eye infection is reduced. The concave shape and drainage through-hole combine to provide a secure seat for the placement of the lens on the apparatus. A small vacuum will be created as the solution drains from underneath the lens, thereby gently securing the lens in place on the hand, allowing the apparatus to be held with the lens in an upright of even upside-down configuration without falling off the hand. This provides for a very useful apparatus that can be handled with relative ease.

In one embodiment of the invention there is provided an apparatus for inserting contact lenses in which the hand is tapered in a chisel cut towards its outermost end. By having the hand tapered into a chisel cut, this will facilitate the hand being inserted underneath the contact lens in the packaging, thereby allowing the contact lens to be picked up easily. In this way, contact with the user's hands is avoided.

In one embodiment of the invention there is provided an apparatus for inserting contact lenses in which the body is thimble shaped for capturing a user's finger therein. This is seen as a very simple construction, simple to manufacture and intuitive to use.

In one embodiment of the invention there is provided an apparatus for inserting contact lenses in which the thimble is open at its outermost end.

In one embodiment of the invention there is provided an apparatus for inserting contact lenses in which the hand is of the order of between 0.008 m (8 mm) and 0.012 m (12 mm) wide at its widest point. This is seen as a useful size to provide a stable base to the lens. Ideally, there is provided an apparatus for inserting contact lenses in which the hand is of the order of 0.01 m (10 mm) wide at its widest point. In one embodiment of the invention there is provided an apparatus for inserting contact lenses in which the drainage through-hole is of the order of between 0.002 (2 mm) and 0.004 (4 mm) in diameter. Ideally, the drainage through-hole is of the order of 0.003 m (3 mm) in diameter. A drainage through-hole of these dimensions will allow a quick release of fluid in which the contact lens is stored from underneath the contact lens as it is picked up, thereby quickly securing the contact lens in position.

In one embodiment of the invention there is provided an apparatus for inserting contact lenses in which the drainage through-hole tapers inwardly from the inner surface of the hand towards an outer surface of the hand.

In one embodiment of the invention there is provided an apparatus for inserting contact lenses in which the drainage through-hole is of the order of 0.0005 m (0.5 mm) wide in diameter at its narrowest point.

In one embodiment of the invention there is provided an apparatus for inserting contact lenses in which the body is constructed from a medical grade silicone. Medical grade silicone is seen as particularly useful as it is relatively simple to mould and furthermore can be sanitized between uses without negatively impacting the material properties of the apparatus.

In one embodiment of the invention there is provided an apparatus for inserting contact lenses in which the medical grade silicone has a shore A hardness of greater than 5 and less than or equal to 35. It is envisaged that a medical grade silicone having a shore A hardness of the order of between 6 and 10, preferably of the order of 7, would be particularly suitable. In one embodiment of the invention there is provided an apparatus for inserting contact lenses in which the apparatus is moulded from a single unitary piece. This is seen as a very simple and inexpensive way to manufacture the device.

In one embodiment of the invention there is provided an apparatus for inserting contact lenses in which the arm extends rearwardly along the body in an elongate spine. In this way, the rigidity of the arm and how the arm flexes can be determined in part by the thickness of the spine and without requiring too much additional material to manufacture the device. This provides for an efficient use of resources.

In one embodiment of the invention there is provided an apparatus for inserting contact lenses in which the arm tapers inwardly from the body towards the hand. In this way, the manner in which the arm will bend including how and where the arm will bend as it is brought into contact with the user's eye (with the lens between the eye and the apparatus) can be determined with precision.

In one embodiment of the invention there is provided an apparatus for inserting contact lenses in which the apparatus is a two-part construction having a first part and a second part releasably connectable together, the first part comprising the body configured to be mounted on a user's finger, the second part comprising the resiliently deformable arm having a hand at its outermost end.

In one embodiment of the invention there is provided an apparatus for inserting contact lenses in which the second part comprises an annular collar at its innermost end, the annular collar being in a plane substantially orthogonal to the plane of the hand.

In one embodiment of the invention there is provided an apparatus for inserting contact lenses in which the first part has a circumferential groove formed in the surface thereof for reception of the annular collar of the second part.

In one embodiment of the invention there is provided an apparatus for inserting contact lenses in which the first part has a slot formed therein for reception and capture of an end of the second part therein. In one embodiment of the invention, the end of the second part forms a push fit into the slot formed in the first part.

In one embodiment of the invention there is provided an apparatus for inserting contact lenses in which one of the first part and the second part are provided with a boss and the other of the first part and the second part are provided with a complementary indent for reception of the boss.

### BRIEF DESCRIPTION OF THE DRAWINGS

The invention will now be more clearly understood from the following description of some embodiments thereof given by way of example only with reference to the accompanying drawings, in which:

FIG. 1 is a left-hand side view of the apparatus according to the invention;

FIG. 2 is a right-hand side view of the apparatus of FIG. 1;

FIG. 3 is front view of the apparatus of FIG. 1;

FIG. 4 is a rear view of the apparatus of FIG. 1;

FIG. 5 is a bottom plan view of the apparatus of FIG. 1;

FIG. 6 is a perspective view of the apparatus of FIG. 1;

FIG. 7 is an enlarged view of portion of the apparatus of FIG. 1;

FIG. 8 is an enlarged view of portion of the apparatus of FIG. 1;

FIG. 9 is an enlarged side view of portion of the apparatus of FIG. 1;

FIG. 10 is an enlarged side view, similar to FIG. 9, with the arm deflected;

FIG. 11 is an enlarged side view of portion of the apparatus of FIG. 1;

FIG. 12 is an enlarged side view, similar to FIG. 11, with the arm deflected;

FIG. 13(a) to 13(d) are views of the apparatus according to the invention in the process of engaging a contact lens;

FIG. 14(a) to 14(f) are views of the apparatus according to the invention engaging a contact lens;

FIG. 15(a) and 15(b) are enlarged views of the apparatus of FIG. 1 engaging a contact lens;

FIG. 16 is a front view of an alternative embodiment of apparatus according to the invention;

FIG. 17 is a rear view of the alternative embodiment of apparatus shown in FIG. 16; and

FIG. 18 is an exploded view of the alternative embodiment of apparatus shown in FIGS. 16 and 17.

### DETAILED DESCRIPTION OF THE DRAWINGS

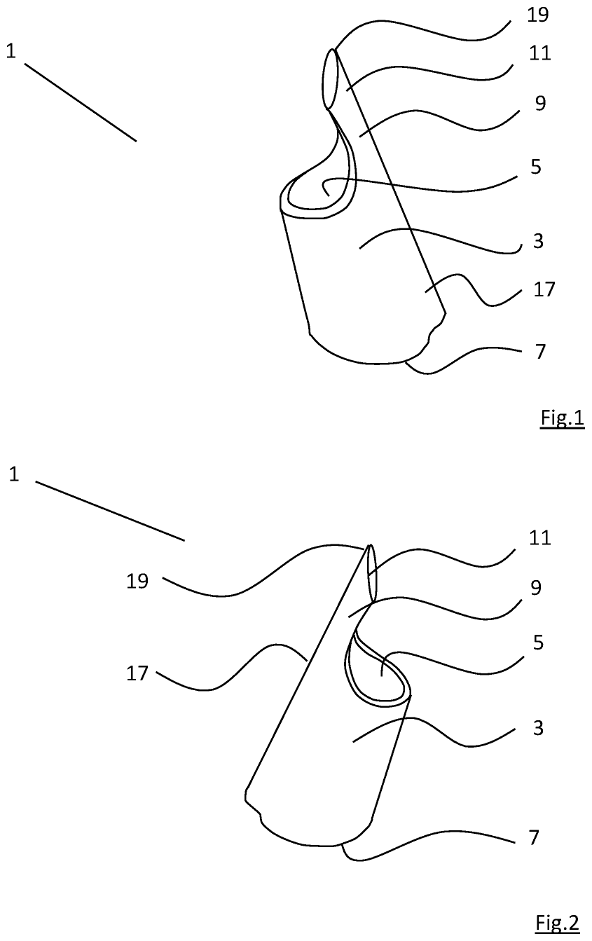

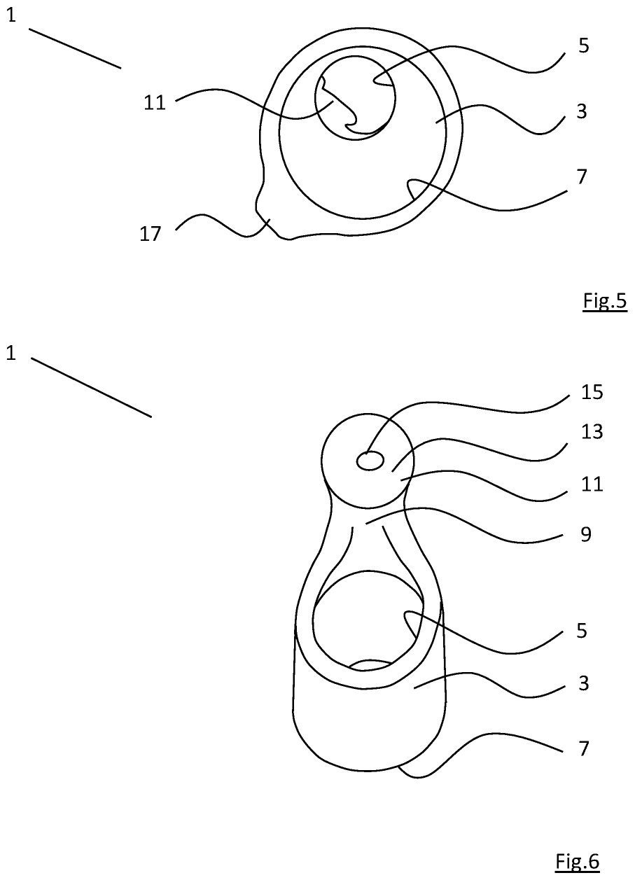

Referring to FIGS. 1 to 8 inclusive, there is shown an apparatus for inserting contact lenses, indicated generally by the reference numeral 1, comprising a thimble-shaped body 3 open at each of its ends 5, 7 and dimensioned to fit onto the end of a user's finger (not shown). The body is constructed from a resiliently deformable material and has an arm 9 extending outwardly therefrom with a hand 11 at the distal end of the arm 9.

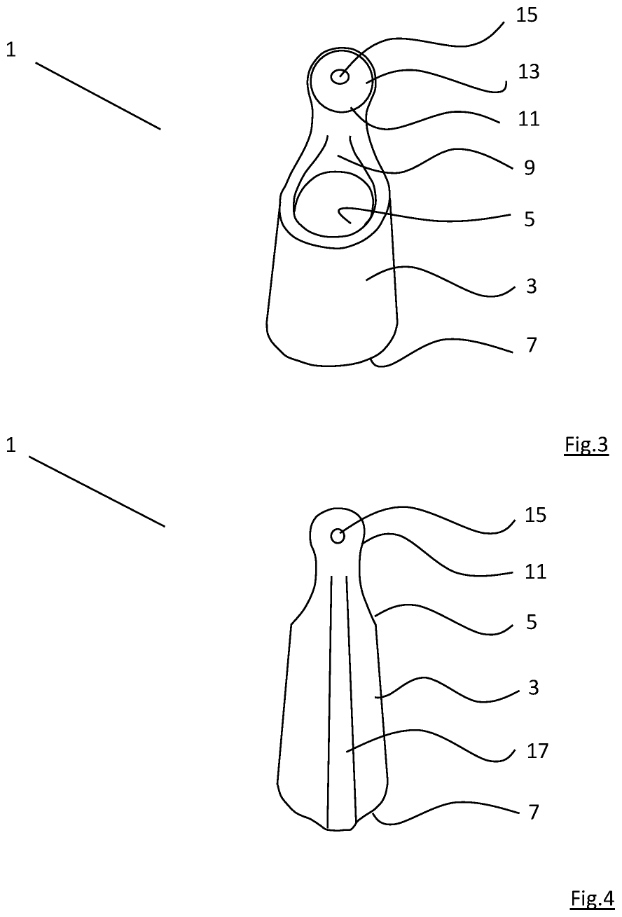

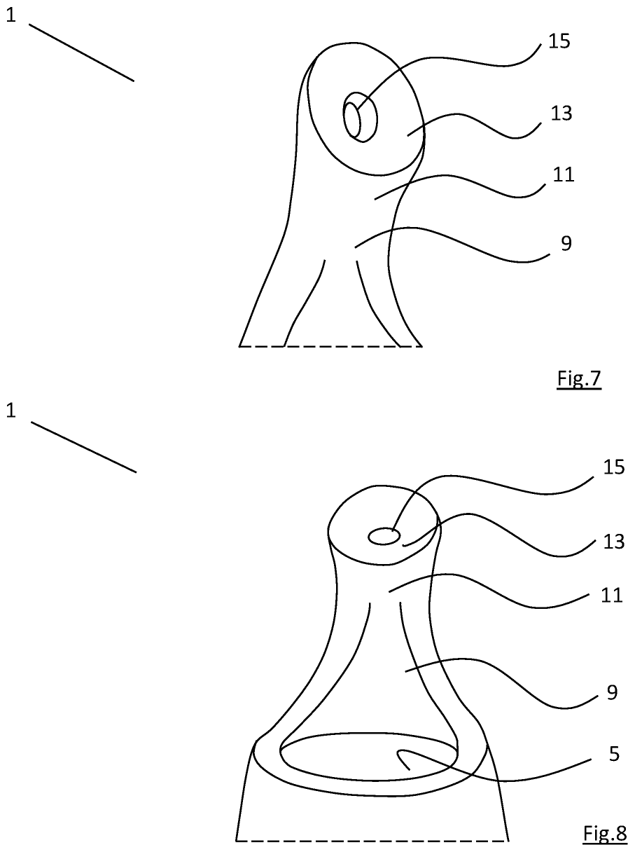

Referring specifically to FIGS. 7 and 8, the hand 11 has a substantially concave inner surface 13 with a through-hole 15. Referring specifically to FIGS. 1 and 2, the body and arm have a spine 17, the thickness of which is determined to provide the desired amount of flexibility to the arm 9 and to ensure that the arm bends in the desired location. In addition, it can be seen that the hand 11 tapers inwardly in a chisel cut towards it's tip 19 at its outermost end. Referring specifically to FIGS. 3 and 4, it can be seen that the arm also tapers inwardly from the point where it contacts the body 3 towards the hand 11.

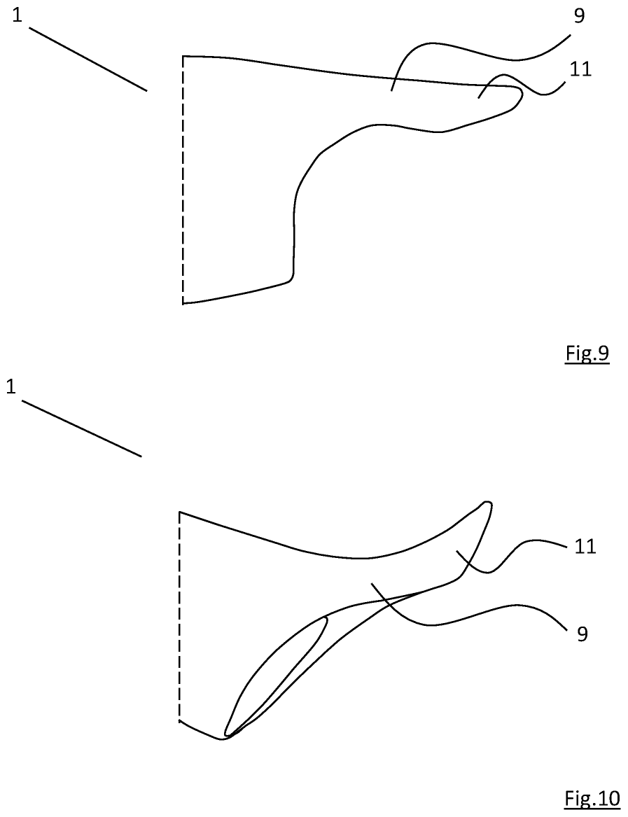

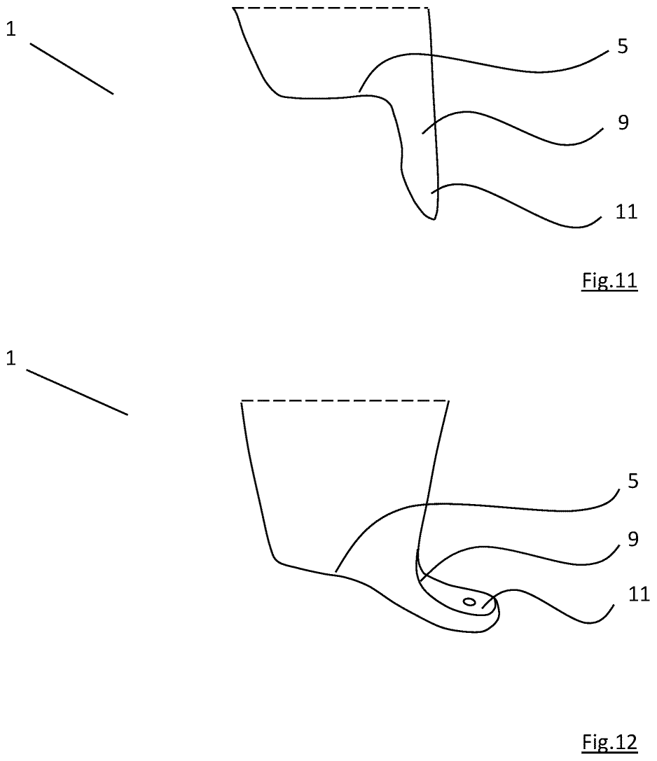

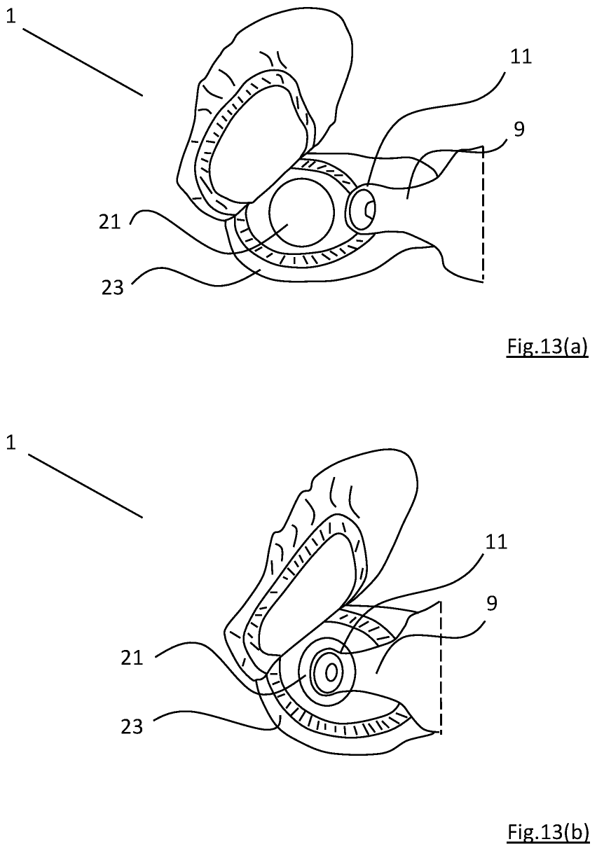

Referring now to FIGS. 9 to 12 inclusive, there are shown views (FIGS. 9 and 11) with the arm at rest, and views (FIGS. 10 and 12) with the arm bent upwards, deflected out of the rest configuration.

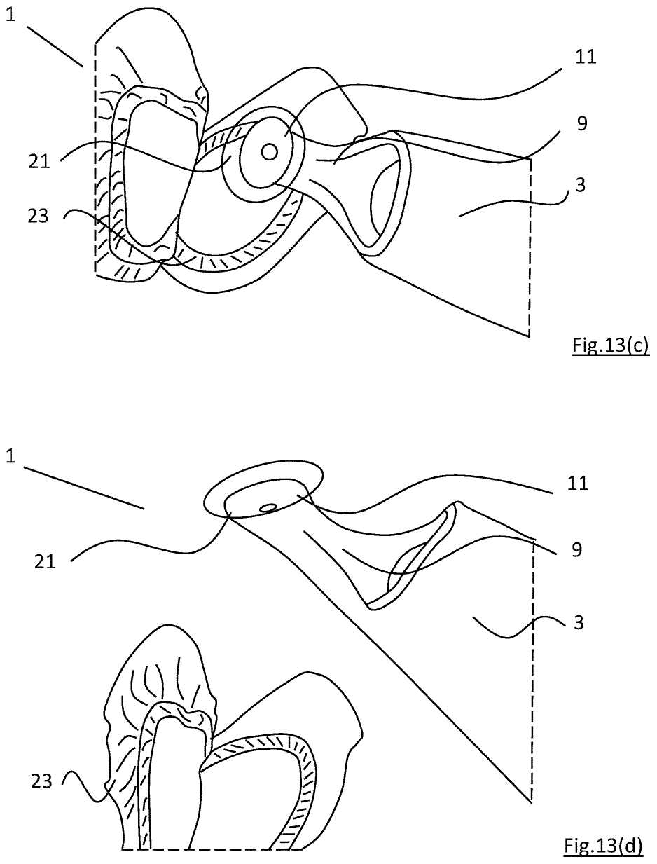

Referring to FIG. 13(a) to 13(d) inclusive, there are shown views of a contact lens 21 being picked up by the apparatus according to the invention. In FIG. 13(a), the user opens the packet 23 containing the contact lens, which in turn is at least partially submerged in a solution in the package. The tip 19 of the hand is gently introduced into the package with the underside of the head sliding along the packaging. In FIG. 13(b), the hand has been advanced to a position underneath the contact lens 21. The apparatus is then lifted upwardly, as shown in FIG. 13(c) and 13(d) where the contact lens is seated in the concave face of the hand, ready for placement in an eye.

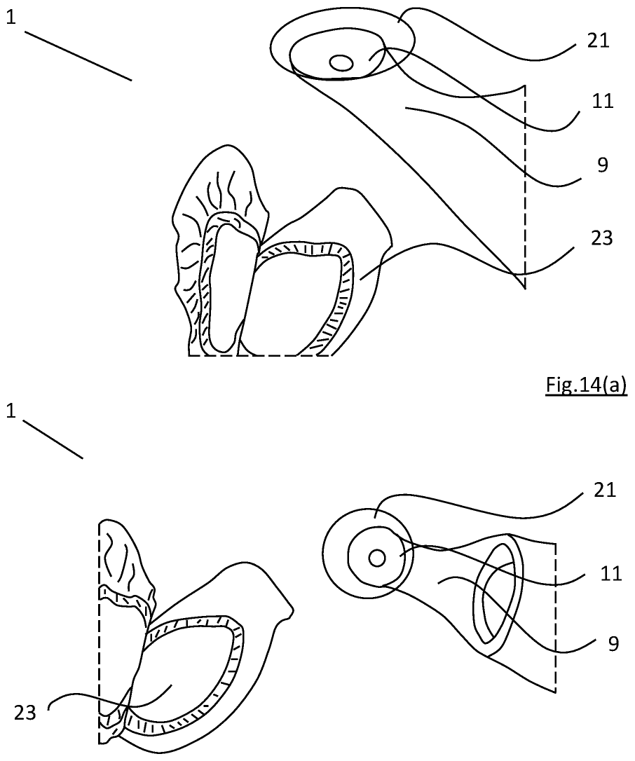

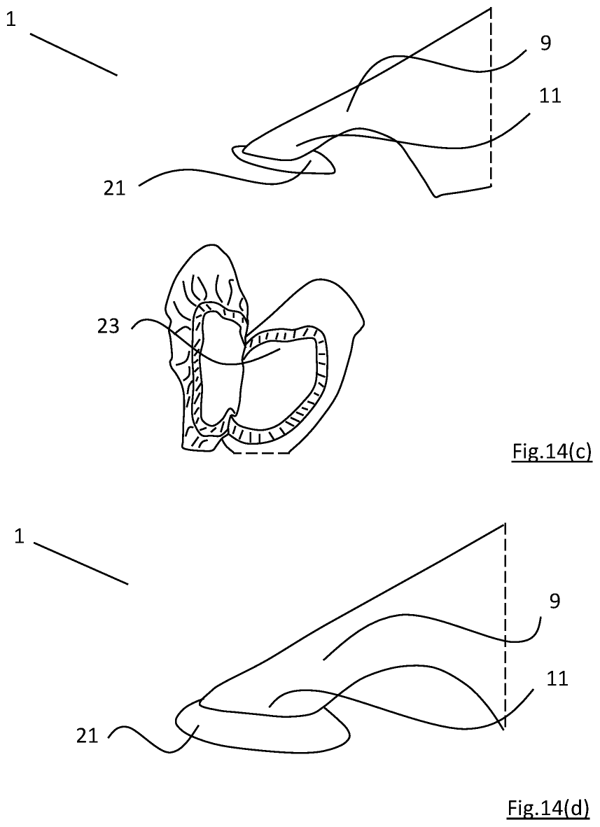

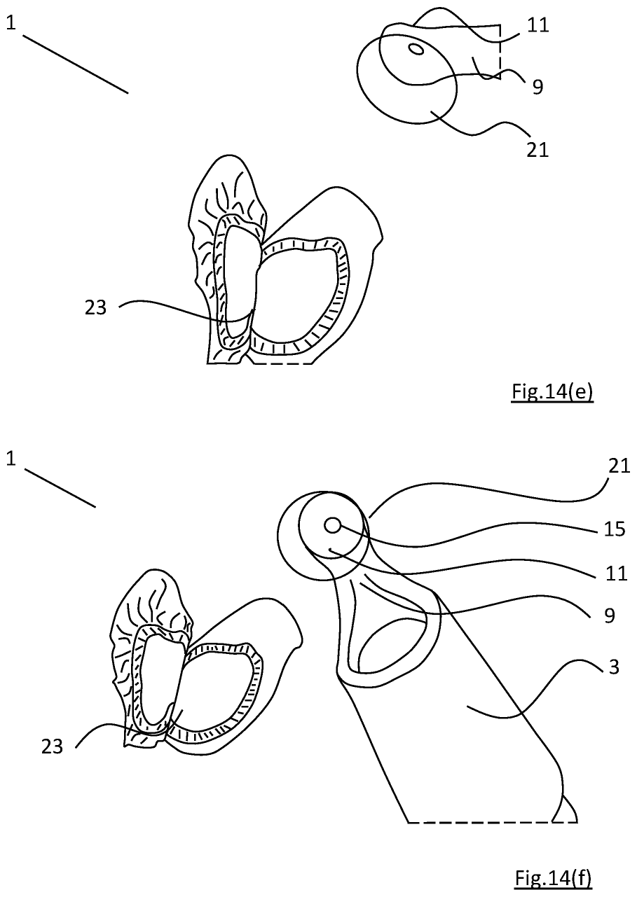

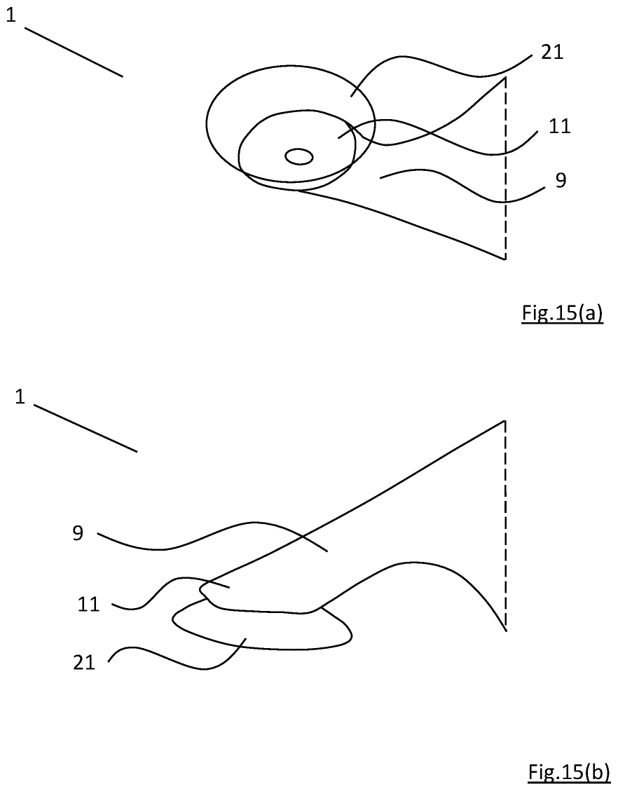

Referring to FIG. 14(a) to 14(f) inclusive, there are shown views of the contact lens 21 being manipulated on the hand 11 of the apparatus. in FIG. 14(a), the contact lens has been removed from the package and sits on the concave inner surface 13 of the hand 11. The contact lens solution underneath the contact lens 21 at least partially drains away through the drainage through-hole 15, thereby creating, it is understood, a miniature vacuum holding the lens in position on the hand 11. In FIG. 14(b), the apparatus has been turned through approximately 60° (with the contact lens at an angle of approximately 60° from the original horizontal configuration) and the contact lens remains in position. In FIG. 14(c), the apparatus has been turned further through approximately 120° with the contact lens at an angle of approximately 120° from the original horizontal configuration and the contact lens remains in position on the hand. Referring to FIG. 14(d), the apparatus has been turned further still through approximately 180° with the contact lens at an angle of approximately 180° from the original horizontal configuration and the contact lens remains in position on the hand, held in place by the small vacuum that was created by the fluid draining out of the drainage through-hole 15. In this position, the apparatus and contact lens have effectively been inverted or turned upside-down. In FIG. 14(e), the apparatus has been turned back through approximately 90° and the contact lens remains in position on the hand, and in FIG. 14(f), the apparatus has been returned to the original position shown in FIG. 14(a) once more, with the contact lens remaining in position.

Referring to FIG. 15(a) and 15(b), there are shown enlarged views of the apparatus 1 according to the invention with a contact lens 21 sitting on the hand 11 of the apparatus. The contact lens 21 is held in place by a small vacuum and/or capillary action and/or surface tension. In use, once the contact lens 21 has been picked up onto the hand 11 of the apparatus 1, the user brings the apparatus up towards their face. If the user has a shaky hand, they may anchor the apparatus by placing the apparatus against their cheek with the hand in alignment with their eye. The user then advances the contact lens 21 towards their eye, positioning the contact lens over the eye. As the user pushes the contact lens against the eye, the arm will deform (bend) and the hand will release the contact lens as pressure is applied to the contact lens pushing it against the eye, and the attractive forces between the contact lens and the eye overcome the forces holding the contact lens to the hand. This is seen as a simple and effective way to insert a contact lens, that will be easy for both the inexperienced and those with unsteady hands to do.

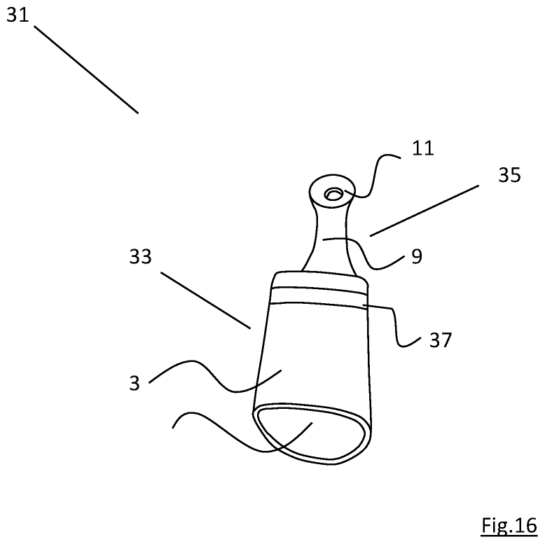

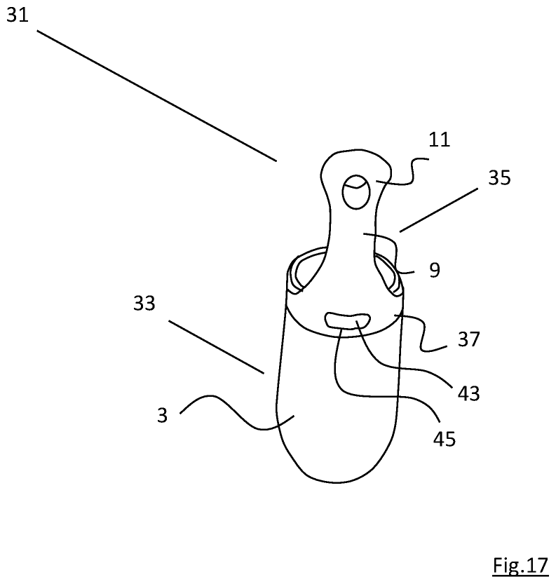

Referring now to FIGS. 16 to 18 inclusive, there is shown an alternative configuration of apparatus for inserting contact lenses according to the invention, indicated generally by the reference numeral 31, where like parts have been given the same reference numeral as before. The apparatus for inserting contract lenses comprises a two-part construction having a first part 33 and a second part 35 releasably connectable together. The first part 33 comprises the thimble-shaped body 3 which is configured to be mounted on a user's finger. The second part 35 comprises the resiliently deformable arm 9 having a hand 11 at its outermost end.

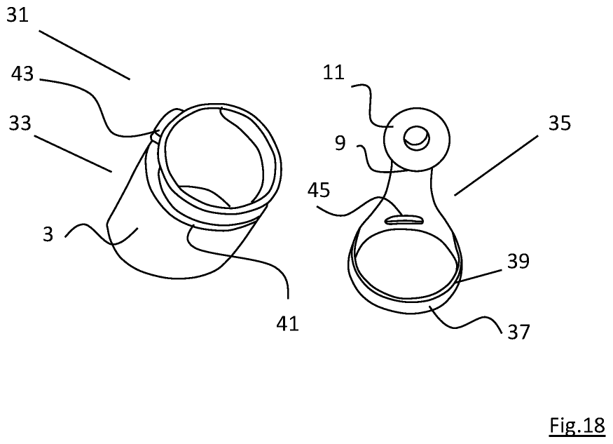

Referring specifically to FIG. 18, there is shown an exploded view of the apparatus for inserting contact lenses with the two parts shown separated from each other. The second part, which comprises the resiliently deformable arm 9 having a hand 11 at its outermost end, further comprises an annular collar 37 at its innermost end 39. The annular collar 37 is in a plane substantially orthogonal to the plane of the hand 11 (and substantially orthogonal to the longitudinal axis of the arm 9). The first part 33 has a circumferential groove 41 formed in the surface thereof for reception of the annular collar 37 of the second part 35. In addition, the first part is provided with a boss 43 and the second part is provided with a complementary indent 45, in this case in the collar portion 37, for reception of the boss. The engagement between the boss 43 and the complementary indent 45 retains the collar, and by extension, the second part, together. It will be understood that other configurations of two-part construction and indeed the manner in which the two parts are connected together are envisaged. For example, the first part may comprise a slot for reception of the end of the second part (i.e. the proximal end of the second part remote from the hand). The proximal end of the second part may form a push fit in the slot on the first part in order to hold the second part in position relative to the first part.

It will be understood that by having the two parts, the two parts may be separated for cleaning, or indeed, one or both parts may be replaced with alternatives or replacement parts over time as they succumb to wear and tear. Alternatively, the first part may be interchangeable with other first parts having different dimensions so that a first part that is particularly suited to a user's finger dimensions may be supplied to the user in each instance. In addition to the foregoing, by having two distinct parts, this will allow for the two parts to be constructed from different materials if desired. The first part may be constructed from a more hardwearing material or a material that fits better on the finger of the user and/or is held securely in place on the wearer's finger. It will however be understood that the second part with the arm and hand will at all times be constructed from medical grade silicone.

As an alternative to the two parts being separable, the two parts may be permanently affixed together with adhesive or other bonding means. Indeed, the boss and complementary indent may be configured so that the boss in dimensioned to remain in the indent unless a great force is exerted on the parts to separate them from each other, such force being practically or effectively sufficient to break one or both parts. More than one boss and complementary indent may be provided. Indeed, there may be one or more bosses and one or more indents on the first part for reception of one or more bosses and one or more indents on the second part (i.e. a mixture of one or more bosses and one or more indents on both of the parts for engagement of complementary indents or bosses respectively on the other part).

In the embodiments shown, the apparatus is constructed from a resiliently deformable material. By that, what is meant is a material that will be able to be bent and/or twisted under an applied force but that will then resume its normal configuration once the force is removed. One suitable material is medical grade silicone. The advantage of medical grade silicone other than having the desired properties is that it is soft and can be sterilized without losing its structural properties. Other elastomers and materials could be used instead. The material will preferably have a shore A hardness of between 5 and 35. It is envisaged that a Shore A hardness of 7 would be suitable.

In the embodiments shown, the hand is of the order of between 0.008 m (8 mm) and 0.012 m (12 mm) wide. Preferably, the hand is of the order of 0.01 m (10 mm) wide. Furthermore, in the embodiments shown, the drainage through-hole is of the order of between 0.002 m (2 mm) and 0.004 m (4 mm) wide. Preferably, the drainage through-hole is of the order of 0.003 m (3 mm) wide. The circumference of the drainage through-hole is circular however this need not necessarily be limiting and other shapes of through-hole are envisaged. For example, rather than having a circular circumference, the circumference could be elliptical in shape. However, a single hole approximately 3 mm in diameter provides sufficient grip of the lens to hold the lens in position for fitting. The inner surface 13 of the hand 11 is concave shaped to match to an extent the curvature of the lens. In other words, the curvature of the concave shape does not have to be identical to the curvature of the lens. The hand is dimensioned and designed to fit underneath the contact lens in the contact lens' enclosure. This will allow for easier extraction of the contact lens from the contact lens' packaging. Furthermore, the curvature and/or size of the hand allow users to ensure that the lens is in the correct position before fitting (i.e. the lens is not “inside-out”).

In one embodiment, the drainage through-hole is funnel shaped, in that it will taper inwardly from the concave top surface towards the convex bottom surface. In such an embodiment, it is envisaged that the through-hole will have a diameter of the order of 0.003 m (3 mm) at its open mouth on the concave upper surface and will taper inwardly to an open discharge outlet with a diameter of the order of 0.0005 m (0.5 mm) on the bottom surface.

In another still embodiment, an inner sleeve may be provided in the body portion to provide an appropriate size for a wearer with smaller fingers. The sleeve may be releasably mounted in the body so that it can be used if needed or discarded if not needed.

In the specification, the term “chisel cut” is used to describe the configuration of the end of the hand. By way of explanation, this will be understood to mean that the hand tapers inwardly to a narrow edge at the tip of the hand from a wider portion remote from the tip of the hand. This enables the tip of the hand to be inserted underneath the contact lens with relative ease. In the embodiment shown, the hand portion is generally circular in shape when viewed from above and concave for reception of the lens. Rather than being circular, the hand portion could be elliptical in shape when viewed from above, akin to a spoon shape.

In this specification, reference is made to medical grade silicone. It will be understood that what is intended is non-implantable medical grade silicone that accords to one or both of ISO 10993 and/or USP Class VI standards. The range of shore A hardness have been mentioned elsewhere throughout the specification however it is envisaged that the shore A hardness will be greater than 5 and less than or equal to 35. The shore A hardness of the hand portion in particular could be of the order of between 6 and 10, preferably of the order of 7.

In this specification the terms “comprise, comprises, comprised and comprising” and the terms “include, includes, included and including” are all deemed interchangeable and should be afforded the widest possible interpretation.

The invention is not limited solely to the embodiments hereinbefore described but may be varied in both construction and detail within the scope of the appended claims.

1) An apparatus for inserting contact lenses comprising a body configured to be releasably mounted on a user's finger, the body having a resiliently deformable arm extending outwardly from a distal end of the body, the arm having a hand at its outermost end, the hand having an inner surface that is concave shaped and defining a drainage through-hole extending through the hand.

2) The apparatus for inserting contact lenses as claimed in claim 1 in which the hand is tapered in a chisel cut towards its outermost end.

3) The apparatus for inserting contact lenses as claimed in claim 1 in which the body is thimble shaped for capturing a user's finger therein.

4) The apparatus for inserting contact lenses as claimed in claim 3 in which the thimble is open at its outermost end.

5) The apparatus for inserting contact lenses as claimed in claim 1 in which the hand is of the order of between 0.008 m (8 mm) and 0.012 m (12 mm) wide at its widest point.

6) (canceled)

7) The apparatus for inserting contact lenses as claimed in claim 1 in which the drainage through-hole is of the order of between 0.002 (2 mm) and 0.004 (4 mm) in diameter.

8) (canceled)

9) The apparatus for inserting contact lenses as claimed in claim 1 in which the drainage through-hole tapers inwardly from the inner surface of the hand towards an outer surface of the hand.

10) The apparatus for inserting contact lenses as claimed in claim 9 in which the drainage through-hole is of the order of 0.0005 m (0.5 mm) wide in diameter at its narrowest point.

11) The apparatus for inserting contact lenses as claimed in claim 1 in which the body is constructed from a medical grade silicone.

12) The apparatus for inserting contact lenses as claimed in claim 11 in which the medical grade silicone has a shore A hardness of greater than 5 and less than or equal to 35.

13) The apparatus for inserting contact lenses as claimed in claim 11 in which the medical grade silicone has a shore A hardness of the order of 7.

14) The apparatus for inserting contact lenses as claimed in claim 1 in which the arm extends rearwardly along the body in an elongate spine.

15) The apparatus for inserting contact lenses as claimed in claim 1 in which the arm tapers inwardly from the body towards the hand.

16) The apparatus for inserting contact lenses as claimed in claim 1 in which the apparatus is moulded from a single unitary piece.

17) The apparatus for inserting contact lenses as claimed in claim 1 in which the apparatus is a two-part construction having a first part and a second part releasably connectable together, the first part comprising the body configured to be mounted on a user's finger, the second part comprising the resiliently deformable arm having a hand at its outermost end.

18) The apparatus for inserting contact lenses as claimed in claim 17 in which the second part comprises an annular collar at its innermost end, the annular collar being in a plane substantially orthogonal to the plane of the hand.

19) The apparatus for inserting contact lenses as claimed in claim 17 in which the first part has a circumferential groove formed in the surface thereof for reception of the annular collar of the second part.

20) The apparatus for inserting contact lenses as claimed in claim 17 in which the first part has a slot formed therein for reception and capture of an end of the second part therein.

21) The apparatus for inserting contact lenses as claimed in claim 18 in which the end of the second part forms a push fit into the slot formed in the first part.

22) The apparatus for inserting contact lenses as claimed in claim 17 in which one of the first part and the second part are provided with a boss and the other of the first part and the second part are provided with a complementary indent for reception of the boss.
